(19) United States
(12) Patent Application Publication
(10) Pub. No.: US 2004/0133209 A1
(43) Pub. Date: Jul. 8, 2004

(54) Medical bur

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/666,501

(22) Filed: Sep. 19, 2003

Related U.S. Application Data
(60) Provisional application No. 60/411,967, filed on Sep. 19, 2002.

(51) Int. Cl. A61B 17/16

(52) U.S. Cl. 606/80

(57) ABSTRACT

A medical bur comprises a bur head comprising a cutting portion being arranged and configured to rotate at least one controllable pre-determined speed and a stop portion being arranged and configured to move independently with respect to the cutting portion.

### CROSS-REFERENCE TO RELATED APPLICATION

This application is based on and claims priority to co-pending U.S. provisional patent application entitled “Medical Bur” filed on Sep. 19, 2002 and accorded serial No. 60/411,967, which is entirely incorporated by reference.

### TECHNICAL FIELD

The invention generally relates to systems and developments related to surgical instruments and, more particularly, relates to medical burs.

### DESCRIPTION OF RELATED ART

High-speed rotational cutting burs are used in various medical applications. Generally, cutting burs can be used to cut through hard biological matter, such as bone, during surgery or other medical procedures. Typical surgical burs include a cutting head mounted toward one end of a shaft. The shaft can be releasably attached to a handle that supplies power to rotate the bur, typically at a high rate of speed. Various bur/shaft combinations are typically interchangeable with any given handle. As such, the bur shape and size is interchangeable to one handle. During any given surgical procedure, the bur may be used in close proximity to exposed soft tissue, such as nerve, muscle, or the like. For example, surgical cutting burs are often used during spinal surgery to cut into a vertebral body. In this application, the high speed rotating cutting bur is often used in close proximity to the spinal cord. The spinal cord itself is soft and fragile and is surrounded by a thin protective film called the dura. Misplacement or slippage of the bur during use in that environment can result in a detrimental outcome for the patient.

Thus, a heretofore unaddressed need exists in the industry to address the aforementioned deficiencies and inadequacies.

### SUMMARY

Medical burs are provided. An embodiment of a medical bur, comprises a bur head comprising a cutting portion being arranged and configured to rotate at at least one controllable pre-determined speed and a stop portion being arranged and configured to move independently with respect to the cutting portion.

Other systems, methods, features and/or advantages will be or may become apparent to one with skill in the art upon examination of the following drawings and detailed description. It is intended that all such additional systems, methods, features and/or advantages be included within this description, and be protected by the accompanying claims.

### BRIEF DESCRIPTION OF THE DRAWINGS

Many aspects can be better understood with reference to the following drawings. The components in the drawing are not necessarily to scale. Moreover, in the drawings, like reference numerals designate corresponding parts throughout the views.

FIG. 1 is a plan view of a preferred embodiment of a medical bur.

### DETAILED DESCRIPTION OF THE PREFERRED EMBODIMENTS

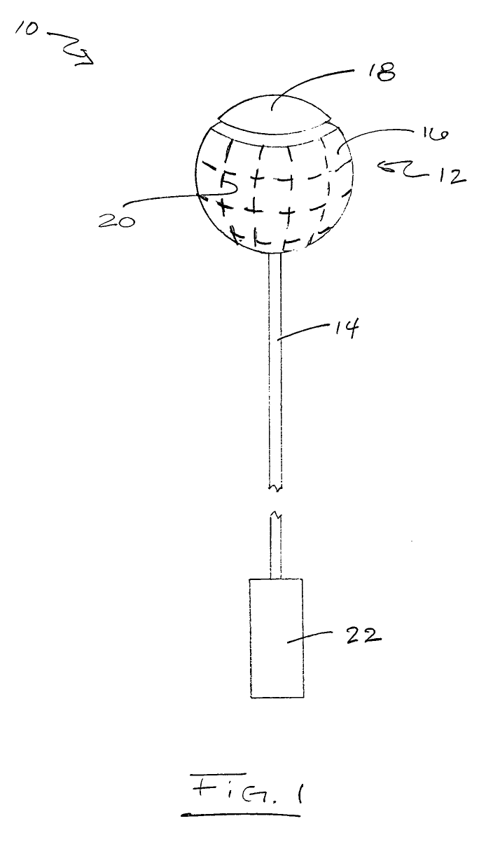

FIG. 1 illustrates an embodiment of a medical bur 10. The medical bur 10 comprises a bur head 12 and a shaft 14. The bur head 12 preferably comprises at least a cutting portion 16 and a stop portion 18. In a preferred embodiment, the cutting portion 16 occupies approximately two-thirds of the volume of the bur head 12. It should be understood, however, that a cutting portion 16 may occupy more or less than two-thirds of the volume of the bur head 12. The cutting portion 16 comprises a cutting surface 20. The cutting surface 20 can comprise any suitable textured surface, for example a grooved surface, a pitted surface, or the like.

Another portion of the volume of the bur head 12 comprises the stop portion 18. The stop portion 18 comprises a substantially smooth surface. It is preferred that the stop portion 18 is a separate member from the cutting portion 16. It should be noted that although the bur head 12 is illustrated as being substantially spherical in shape, the bur head 12 can be formed in many different shapes such as, but not limited to, barrel-shaped, elliptical, acorn-shaped, or any other suitable shape.

The bur head 12 is supported by the shaft 14 extending from the cutting portion 16 of the bur head 12. A handle 22 is disposed on the shaft 14 opposing the bur head 12. A preferred shaft 14 is arranged and configured to provide distance between the bur head 12 and a handle 22 and to rotate the cutting portion 16 of the bur head 12. The handle 22 releasably receives the shaft 14. The handle 22 can supply power to the shaft 14 and/or bur head 12 for rotation. Power can be supplied by batteries, DC power, or any suitable source. The handle 22 is also grasped by the user of the medical bur 10 during use. The bur head 12 can be supported by any suitable member that can provide power to a portion of the bur head 12 and free rotation to another portion of the bur head 12. Likewise, power can be supplied to the portion of the bur head 12 arranged and configured for power rotation by any suitable power supply.

The cutting portion 16 of the bur head 12 is arranged and configured to rotate at a desired speed and is powered by a power supply. The stop portion 18 is fixed to the cutting portion 16 and/or the shaft 14 in a freely rotational manner. As such, the stop portion 18 is substantially free-moving about the axis of connection in a rotational direction. It is preferable that the stop portion 18 does not rotate under power but instead is free to move in a rotational direction as necessary upon coming into contact with a material, such as but not limited to soft tissue. In the event that the bur head 12 contacts soft tissue as a result of misplacement or slippage while the cutting portion 16 is utilized, the stop portion 18 contacts the soft tissue rather than the cutting portion 16 making contact. The free-moving rotational configuration allows the stop portion 18 to absorb some of the force of the contact with the soft tissue, thereby reducing or eliminating damage to the soft tissue.

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claim.

Therefore, having thus described the invention, at least the following is claimed:

1. A medical bur, comprising:
a bur head having a cutting portion and a stop portion; and
a shaft extending from said bur head;
wherein said cutting portion of said bur head is adapted to rotate under power and said stop portion of said bur head is adapted to move independently with respect to said cutting portion.

2. The medical bur of claim 1, wherein said stop portion is arranged and configured to rotate freely.

3. A bur head of a medical bur, wherein said bur head comprises:
a cutting portion being arranged and configured to rotate at at least one controllable pre-determined speed; and
a stop portion being arranged and configured to move independently with respect to said cutting portion.

4. The medical bur of claim 3, wherein said stop portion is arranged and configured to rotate freely.
